(19) United States
(12) Patent Application Publication
(10) Pub. No.: US 2022/0042106 A1
(43) Pub. Date: Feb. 10, 2022

(54) SYSTEMS AND METHODS OF USING CELL-FREE NUCLEIC ACIDS TO TAILOR CANCER TREATMENT

(71) Applicant: Agendia NV, Amsterdam (NL)

(72) Inventors: Bastiaan Van Der Baan, Irvine, CA (US); Annuska Maria Gias, Irvine, CA (US)

(21) Appl. No.: 17/395,011

(22) Filed: Aug. 5, 2021

Related U.S. Application Data
(60) Provisional application No. 63/062,111, filed on Aug. 6, 2020.

(51) Int. Cl. C12Q 1/6886; G16B 25/10; G16B 40/00; G16H 50/20; G16H 70/60; G16H 20/40

(52) U.S. Cl. CPC C12Q 1/6886; G16B 25/10; G16B 40/00; C12Q 2600/106; G16H 70/60; G16H 20/40; C12Q 2600/158; G16H 50/20

(57) ABSTRACT

This disclosure relates to systems and methods for assessing disease from cell-free nucleic acids to tailor a treatment. In particular, the systems and methods described herein identify cell-free nucleic acids from a body fluid sample and use the identified cell-free nucleic acids to produce expression signatures that are indicative of disease severity. The expression signatures are correlated with known outcomes to provide prognostic information for the patient, thereby allowing a clinician to tailor a treatment to a predicted disease severity.

### TECHNICAL FIELD

The present invention relates to oncology. More particularly, the present invention relates to systems and methods for tailoring a cancer treatment using cell-free nucleic acids.

### BACKGROUND

Breast cancer patients with the same stage of disease can have markedly different treatment responses and outcomes. Some of the strongest predictors for recurrence and spread of cancer (metastasis), such as, lymph node status and histological grade, often fail to identify patients that need chemotherapy.

For example, clinicians often recommend chemotherapy following the excision of a tumor to prevent cancer recurrence and metastasis. Chemotherapy is a systemic treatment of highly toxic drugs that travel throughout the body killing cancer cells. Unfortunately, chemotherapy kills many healthy cells too, often causing severe side effects including nerve damage, heart failure, and leukemia.

However, only a fraction of cancer patients benefits from chemotherapy. Many patients are at such a low risk for recurrence or metastasis that chemotherapy is unnecessary. Unfortunately, clinicians cannot easily distinguish which patients will and will not benefit from chemotherapy treatment. And as such, many patients are over treated and must unnecessarily suffer from harsh and expensive drugs that often lead to severe health consequences.

### SUMMARY

The invention relates to assessments of disease using nucleic acids released from tumor cells to provide patient-specific cancer treatment. The nucleic acids (preferably cell-free nucleic acids) are measured from a body fluid sample to create one or more grouped expression signatures. The expression signatures reflect the genes that are expressed in the cells of the tumor and are useful for assessing disease severity. In particular, expression signatures may be correlated with expression signatures of known treatment outcomes to produce prognostic information for tailoring treatment. For example, correlations with known outcomes are used to identify patients who may certain chemotherapies and associated toxicity. In addition, signatures are useful to identify optimal treatment regimens, including therapeutic selection.

Methods of the invention provide an avenue for non-invasive cancer management by utilizing cell-free nucleic acids from tumors. Moreover, methods of the invention are useful for longitudinal disease management and assessment of treatment efficacy without resorting to invasive procedures. For example, analysis of cell-free nucleic acids, e.g., DNA or RNA, can be done prior to biopsy or surgical resection and then again at any time or times post extraction in order to assess disease progression, regression, recurrence or residual disease. In other instances, methods of the invention may be used to assess the efficacy of a therapy in a cancer patient. In other instances, the expression signatures may be useful for classifying patient and selecting an optimal therapeutic.

In one aspect, the invention provides methods in which at least two cell-free nucleic acids in a body fluid sample from a patient are grouped based on their positive predictive value for disease severity. The groupings then are used as a diagnostic marker to assess disease severity. Combinations of nucleic acid markers, once correlated with predictive value, can be used to assess new patients or can be used to assess the clinical status of the patient from whom they were obtained, depending on the universality of the detected mutations with respect to a particular cancer. In preferred embodiments, the invention further provides for selecting a course of treatment for the patient. The invention allows for screening patients to determine which patients are good candidates for chemotherapy and which patients may be able to avoid chemotherapy entirely or partially.

Systems and methods of the invention are used to predict how well an individual will respond to certain treatments. Thus, treatment selection can be tied to outcome based on the predictive value of the combined groups of cell-free nucleic acid. The invention allows intervention at an early stage of disease with positive predictive value for treatment. For example, in diseases such as cancer, early intervention with the right treatment provides an increased probability of a positive treatment outcome.

Groups of cell-free nucleic acid with high correlation to disease outcome are themselves drivers of therapeutic selection. According to the invention, drug options are correlated with signatures obtained through methods described and claimed herein.

Methods of the invention are useful to analyze cell-free nucleic acids taken from a body fluid sample to assess cancer. The body fluid sample may be blood, saliva, sputum, urine, semen, transvaginal fluid, cerebrospinal fluid, sweat, stool, or any other bodily fluid or secretion Preferably, the body fluid sample is blood, as it is an insight of the invention that cell-free nucleic acids are surprisingly stable in blood when encapsulated inside extracellular vesicles where they are protected from degradation.

Cell-free nucleic acids include DNA and RNA, but RNA, and more preferably, messenger RNA (mRNA) is preferred. The mRNA may include, for example, one or more transcripts from oncogenes. For example, there are known oncogenes associated with breast cancer and known to the skilled artisan. The mRNA may comprise transcripts used in diagnostic cancer assays, such as the cancer assays sold under trade names MammaPrint and/or BluePrint by Agendia, Inc., which are able to distinguish patients that are either low risk or high risk of distant metastasis that and assess the molecular subtype of breast cancer.

Both the types and amounts of cell-free nucleic acid are diagnostic with respect to drug treatment options, predictive survival rates and other aspects of disease management. Combinations of cell-free nucleic acids increase the positive predictive value of the diagnostic with respect to, for example, 5-year survival rates. The cell-free nucleic acids may comprise gene transcripts that are associated with histopathological data, for example, the transcripts may arise from genes may are associated with oestrogen receptor (ER)-alpha.

Methods of the invention may further include measuring quantities of cell-free nucleic acids, e.g., mRNA, and using the measured quantities, which may be weighted quantities, to determine expression levels for distinct species of mRNA. Preferably, methods of the invention involve making a next generation sequencing library for sequencing.

Certain methods comprise using target enrichment next-generation sequencing technologies to detect specific species of mRNAs. Advantageously, this allows researchers and clinicians to focus analyses on specific mRNAs of interest, such as, mRNA with positive predictive value for disease outcome, thereby eliminating time and expenses wasted on processing material that is of little value. For example, methods of the invention may involve probing mRNA associated with a panel of genes and measuring quantities of mRNA associated with the gene panel. The mRNA may be derived from a panel of genes involved in hormone receptor regulation. The mRNA may be derived from the panel of genes associated with diagnostic breast cancer tests MammaPrint and/or BluePrint by Agendia, Inc.

A preferred method of the invention comprises creating a cDNA copy of each mRNA molecule and then sequencing the cDNA copies to generate a plurality of sequencing reads. Sequencing may be accomplished using any standard sequencing technology. The sequencing reads may be analyzed to determine expression levels of distinct species of mRNA. Determining expression levels preferably involves mapping the sequence reads to a reference genome and counting reads that map to each locus. Determined expression levels are then used to create patient-specific expression signatures. Preferably, the expression signatures include only those species of mRNAs that are expressed at levels substantially above a level that is associated with background noise.

Methods of the invention may include analyzing an image from a stained tissue sample to support of confirm a disease assessment made from cell-free nucleic acids. For example, the image may be an image of a tumor sample from the patient and stained with, for example, H&E stain, Pap stain, an immunohistochemical stain, or any other suitable staining/labelling media. The staining may reveal specific molecular markers that are indicative of disease stage and progression. For example, immunohistochemistry staining may be used to reveal intracellular proteins characteristic of a tumor. Accordingly, methods of the invention include obtaining an image of a stained tissue sample from a patient; and analyzing the image to detect one or more features indicative of disease severity to support or confirm a prognosis or selected treatment.

In some instances, the invention may exploit the correlative powers of an analysis system, such as a machine learning system, to assess disease. For example, an analysis system may be used to autonomously predict treatment responses or disease severity based on learned associations from training data. Methods may include providing expression data from a patient as an input to an analysis system trained on training data comprising one or more sets of training expression level measurements associated with known patient outcomes. Preferably, the analysis system comprises a computer system with a machine learning algorithm. The analysis system may be a machine learning system. Using the power of machine learning, the methods and systems of the invention can leverage vast amounts of old and/or new data to provide more accurate and patient-specific diagnoses, prognoses, and treatment suggestions.

Further, other data, such as image data from the patient, may be provided as part of the inputs to the analysis system. The methods and systems of the disclosure can analyze this disparate data, such as expression levels of nucleic acids and image data, in combination, to provide correlative diagnoses, prognoses, and treatment suggestions. The methods and systems of the disclosure may include an analysis system hosting a trained machine learning algorithm. Image data provided as an input may be an image of a stained, FFPE slide from a tumor from the patient.

Further provided are methods of preparing nucleic acid libraries for sequencing to predict the prognosis or response to therapy of a subject diagnosed with or suspected of having breast cancer. These methods are useful for creating sequencing libraries, which after sequencing, may be analyzed according to methods described herein to guide or determine treatment options for a subject suffering from breast cancer. The methods of the invention further include kits comprising means for assessing expression of cell-free nucleic acids.

### BRIEF DESCRIPTION OF THE DRAWINGS

FIG. 1 diagrams a method for assessing disease.

FIG. 2 shows a body fluid sample.

FIG. 3 diagrams a method of sample prep.

FIG. 4 shows an analysis system.

### DETAILED DESCRIPTION

This disclosure relates to systems and methods for assessing disease from cell-free nucleic acids to predict treatment response and disease progression (including the likelihood of metastasis or recurrence or the presence of residual disease). Systems and methods described herein may measure cell-free nucleic acid as a proxy for expression of disease-related genes. The measurements may be used to create one or more expression signatures indicative of disease severity, outcome, or therapeutic selection. In cancer, expression signatures are correlated with expression signatures from tumors associated with known outcomes in order to generate diagnostic and prognostic criteria that allows management of future patients with the same or similar signature. For example, methods of the invention are useful to identify a patient who may safely avoid chemotherapy and/or may be used to guide a course of treatment by identifying a drug that will be effective for treating the cancer.

Preferably, the cell-free nucleic acids are obtained from a blood sample so that patients can be monitored over time to assess disease progression and therapeutic effectiveness. For example, patients may be evaluated before and/or after a tumor is removed to determine whether the patient's tumor is likely to recur and/or metastasize, which may indicate that the patient will benefit from one or more rounds of chemotherapy. In other instances, methods of the invention are used to assess cancer in a patient undergoing chemotherapy to determine whether the patient is responding to the chemotherapy treatment and whether additional chemotherapy treatments are within the patient's best interest. In other instances, methods of the invention are useful for selecting a drug to treat the cancer patient. Such as, for example, a drug for use in a chemotherapy treatment.

Chemotherapy, including adjuvant therapy, usually causes side effects, such as nausea, vomiting, loss of appetite, loss of hair, mouth sores, and severe diarrhea. In some instances, the side effects are severe. For example, chemotherapy may lead to nerve damage, heart attacks, or leukemia. For all patients, the risk of cancer recurrence and metastasis should be weighed against the side effects caused by aggressive treatment. Patients with a high risk for cancer recurrence, for example, may benefit from adjuvant therapy, while patients with a low risk will unnecessarily suffer from the severe side effects caused by adjuvant therapy. Systems and methods of the invention offer the unique ability to tailor treatment by predicting a risk of cancer recurrence and metastasis from nucleic acids present in body fluid and evaluating treatment options based on the predicted risk.

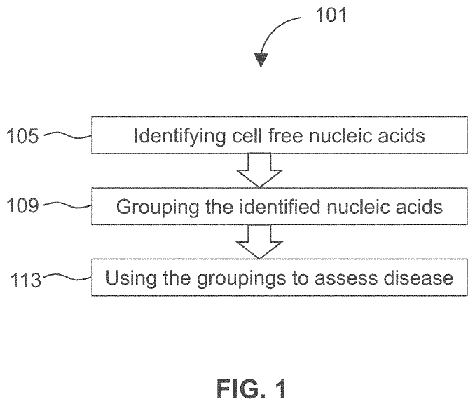

FIG. 1 diagrams a method 101 for assessing disease. The method includes identifying 105 at least two cell-free nucleic acids in a body fluid sample from a patient and grouping 109 the identified nucleic acids based on their positive predictive value for disease severity. The method 101 further includes using 113 one or more of the groupings to assess disease.

Cell-free nucleic acids are identified 105 from a body fluid sample. Because the method 101 of the disclosure can use samples obtained from bodily fluids, testing and analysis is far more rapid than existing tests. Consequently, physicians can quickly administer an appropriate and effective treatment. This helps improve the prognoses of patients with early-stage breast cancer. The body fluid sample may comprise one of blood, saliva, sputum, urine, semen, transvaginal fluid, cerebrospinal fluid, sweat, stool, a cell or a tissue. In preferred embodiments, the sample comprises blood, which may be collected during a routine blood draw.

Preferably, the body fluid sample is collected from a patient that is suspected of having a disease, such as cancer. The patient may be suspected of having a cancer on account of various symptoms including the detection of a lump or mass. The cancer may be one of bladder cancer; breast cancer; colorectal cancer; kidney cancer; lung cancer; lymphoma; skin cancer; oral cancer; pancreatic cancer; prostate cancer; thyroid cancer; or uterine cancer. The method 101 is particularly well suited for assessing patients with breast cancer, which is the preferred embodiment. More preferably, the cancer is early stage breast cancer, i.e., cancer that is contained entirely within the breast.

The body fluid sample may be processed to isolate cell-free nucleic acids using, for example, a commercially available kit, such as the kit sold under the trade name QIAamp Circulating Nucleic Acid Kit by Qiagen. Preferably, the cell-free nucleic acids comprise RNA, and more preferably, the cell free nucleic acids comprise mRNA. The mRNA may include gene transcripts of genes that are differentially expressed in early stage breast cancer to allow for disease assessments. For example, the mRNA may include gene transcripts genes evaluated by MammaPrint and/or BluePrint, for example, as described in U.S. Pat. No. 10,072,301 and WO2002/103320, which are incorporated herein by reference.

The cell-free nucleic acids, e.g., mRNA, may be identified 105, i.e., detected and quantified, by any of a wide variety of methods. Method include, but not limited to, sequencing (e.g., RNA-seq), hybridization analysis, amplification e.g., via the polymerase chain reaction, for example, by reverse transcription polymerase chain reaction (RT-PCR). In preferred embodiments, identifying 105 involves targeted enrichment next-generation sequencing technologies, which are useful to identify 105 specific nucleic acids of interest, for example, as described in Mittempergher, 2019, MammaPrint and BluePrint Molecular Diagnostics Using Targeted RNA Next-Generation Sequencing Technology, The Journal of Molecular Diagnostics, Volume 21, Issue 5, 808-823, which is incorporated by reference.

Identifying 105 may involve isolating mRNA from the body fluid sample and uniquely barcoding each molecule of mRNA. The mRNA can be converted into complementary DNA (cDNA). Specific cDNA molecules associated with, for example, any one of the reported MammaPrint and/or BluePrint genes, may be probed for using biotinylated capture RNA baits. The captured cDNA molecules can be analyzed by sequencing to produce a plurality of sequence reads. The plurality of sequence reads may be de-duplicated based on the unique barcodes and mapped to a reference genome to identify their genetic origin. Sequence reads that map to each locus of the reference genome are then counted to determine expression levels of the identified 105 cell-free nucleic acids of interest.

Once the at least two cell-free nucleic acids are identified 105 from the body fluid sample, a portion of the at least two cell-free nucleic acids are grouped 109 together based on their positive predictive value for disease severity.

Grouping 109 based on predictive value for disease severity may involve a clustering algorithm. A clustering algorithm is an algorithm that clusters or groups a set of objects in such a way that the objects in the same group (called a cluster) are more similar to each other than to those in other groups (clusters). The clustering algorithm may be an unsupervised hierarchical clustering algorithm, such as, a K-means clustering algorithm.

The clustering algorithm may be used to cluster expression levels of nucleic acids from tumors with known outcomes. The clusters may reveal patterns of expression that are associated with disease severity based on known the known outcomes. The patterns may comprise nucleic acids associated with genes that are upregulated or downregulated in breast cancer with high statistical significance. For example, one or more patterns of expression may emerge that are associated with a good prognosis, e.g., no recurrence or metastasis of disease. Other patterns of expression may emerge that are associated with a poor prognosis, e.g., recurrence or metastasis of disease. The nucleic acids that correlate highly with an outcome have a positive predictive value for disease. Accordingly, the clustering algorithm may group similarly expressed levels of nucleic acids from tumors together based on their known outcomes to reveal nucleic acids that have positive predictive values for disease severity.

For example, a clustering analysis from breast tumors may reveal that nucleic acids associated with the following genes have positive predictive value for disease such as breast cancer: NPY1R, TPRG1, SUSD3, CCDCl74B, CHAD, GREB1, PARD6B, PREX1, GOLSYN, ACADSB, ADM, SOX11, CDCl25B, LILRB3, and HK3 PRR15, ABCC11, DHRS2, TBC1D9, GREB1, THSD4, CHAD, and PERLD1.

Preferably, grouping 109 cell-free nucleic acids based on positive predictive value for disease severity involves creating one or more expression signatures. An expression signature is combined group of nucleic acids with a uniquely characteristic pattern of expression that occurs as a result of an altered a biological process or pathogenic condition. Preferably, the cell-free nucleic acids that correspond with the nucleic acids found to correlate with an outcome are grouped together to create one or more expression signatures. For example, grouping 109 may comprise selecting one or more of the nucleic acids associated with genes that have positive predictive value for breast cancer, for example, NPY1R, TPRG1, SUSD3, CCDCl74B, CHAD, and GREB1, and creating an expression signature with those genes.

After grouping 109 the cell-free nucleic acids to create one or more expression signatures, the expression signature can be used 113 to assess disease by correlating levels of expression with levels of expression associated with outcomes identified by the clustering algorithm. A high correlation with, for example, a signature associated with a good prognosis may indicate the patient is unlikely to suffer from disease recurrence or residual disease.

The clustering algorithm may be used to distinguish the molecular subtypes, (e.g., Basal-type, Luminal-type, or Her2-type) of the patient tumor. For example, the clustering algorithm may be used to cluster expression levels of nucleic acid expression from tumors associated with known molecular subtypes based on, for example, immunohistochemistry staining. The cell-free nucleic acids that correspond to nucleic acids that positively correlate with a molecular subtype may be grouped together to create an expression signature. The expression signature may then be correlated with the expression signatures of the clustering analysis to identify the molecular subtype of the patient tumor. Identifying the molecular subtype of the cancer may better predict clinical outcome and help determine whether the addition of adjuvant chemotherapy to endocrine therapy is worthwhile.

For example, patients with Her2-type breast cancer may be treated with Trastuzumab. which specifically targets Her2-type. Trastuzumab is often used with chemotherapy but it may also be used alone or in combination with hormone-blocking medications, such as an aromatase inhibitor or tamoxifen. Her2-type patients can also be treated with Lapatinib (Tykerb) in combination with the chemotherapy drug capecitabine (Xeloda) and the aromatase inhibitor letrozole (Femara). Lapatinib is also being studied in combination with trastuzumab. Further therapies may include an AKT inhibitor and/or a Tor inhibitor, either alone or in combination with hormone-blocking medication.

Preferably, the grouping 109 step is only performed with nucleic acids that are expressed at a level that is substantially above a level of expression identified as background noise. For example, in some instances, the grouping 109 step is only be performed with nucleic acids that are expressed at least 1-fold, 2-fold, or 3-fold above a level identified of expression that is as background noise. By grouping 109 only those nucleic acids that are expressed substantially above background noise, the gene expression signatures are more stable and less likely to be impacted by experimental variability.

In some embodiments, expression signatures are used to assess disease severity by correlating the one or more expression signatures with one or more expression signatures of patients with known outcomes. Such correlations may be used to assess likelihood of a distant metastasis event or cancer recurrence. For example, one or more gene expression signature may be identified as being indicative of a low risk of cancer recurrence. This may be based in part on known patient outcomes in which patients presenting similar expression signatures are found to be cancer free 5 years or 10 years after treatment. Accordingly, methods of the invention may involve creating a patient specific expression signature by grouping at least a portion of the identified cell-free nucleic acids and assessing disease by correlating the patient specific expression signature with one or more signatures having a known outcome to make a determination about the patient. For example, if a patient has an expression signature that highly correlates with a signature associated with a first patient that had a cancer recurrence, the patient is at high risk for cancer recurrence. In preferred embodiments, the correlation is performed using a computer algorithm.

The methods of the invention may be used to predict how well a given patient will respond to certain treatments. Because methods of the invention are useful for predicting treatment response, an effective treatment may be recommended to the patient, and clinicians can avoid spending the time and money on treatment protocols that will not help the patient. Recommending a treatment may involve selecting one or more drugs likely to be effective for treating the patient. Because an effective treatment is given to the patient rapidly, the patient with a tumor or an early stage cancer will have a good chance of remission and recovery. Selecting a course of treatment may further involve identifying a drug that a patient is likely to respond to by, for example, determining or predicting a response of the patient to the treatment. In some embodiments, selecting a course of treatment involves determining that the patient does not need to be treated or determining that a patient needs a tumor resection.

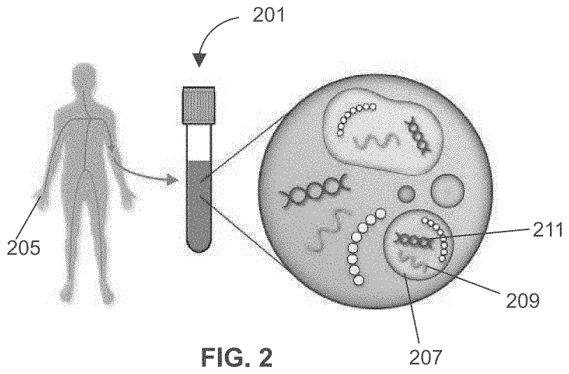

FIG. 2 shows a body fluid sample 201. The body fluid sample 201 comprises blood 203 and is preferably taken from a patient 205 by blood draw. The blood 203 may include extracellular vesicles 207. Extracellular vesicles 207 are small plasma membrane-encapsulated particles, which comprise exosomes and microvesicles, that are released by all cells and that can enter the bloodstream. Extracellular vesicles 207 are ubiquitous in body fluids including blood plasma, cerebral spinal fluid, aqueous humor, amniotic fluid, saliva, synovial fluid, adipose tissue, and urine. Both blood plasma and cerebral spinal fluid extracellular vesicles including exosomes are a useful source of cell-free nucleic acids for assessing disease.

Extracellular vesicles 207 contain proteins (tumor antigens, immunosuppressive, and/or angiogenic molecules) and cell-free nucleic acids, including cell free RNA 209 and cell free DNA 211 specific to cancer cells. Thus, their cargo may be analyzed to determine their cell of origin by, for example, by segregating the extracellular vesicles 207 and sequencing the nucleic acids contained therein or performing an immunochemistry staining for cell-type specific proteins. In some cases, the extracellular vesicles 207 may be segregated by immunostaining the extracellular vesicles 207 for a protein that is over or under expressed in cancer, and subsequently sorting the stained extracellular vesicles 207 by FACS.

Methods of the invention may include determining an extracellular vesicle's origin (e.g., determining that the vesicle was released from a tumor cell) based on the content of the extracellular vesicle before identifying at least two of the cell-free nucleic acids contained therein, as described below. By determining the extracellular vesicle's origin prior to identifying the cell-free nucleic acids, a researcher or clinician, may focus their analyses specifically on nucleic acids associated with tumor cells. Accordingly, methods of the invention allow for the analysis of cargo of extracellular, after those extracellular vesicles have been isolated form a blood or plasma sample form the patient, to thereby track and predict tumor growth.

The extracellular vesicles may be isolated from blood collected by blood draw or by fine needle aspiration. Isolating the extracellular vesicles from the body fluid sample may involve a differential ultracentrifugation (low-speed centrifugation to remove cells and debris, high-speed ultracentrifugation to pellet exosomes). For example, to isolate extracellular vesicles from blood the sample, may be centrifuged at low speeds allowing for the removal of cells and debris by, for example, pipetting or dumping out supernatant. The sample may then be centrifuged at high speeds, for example, at 100,000×g for 70 min, to pellet the extracellular vesicles allowing the extracellular vesicles to be separated from remaining material. Easy-to-use precipitation solutions, such as the precipitation solution sold under the trade name ExoQuick by System Biosciences, may be used to precipitate the vesicles in liquid. Once the vesicles are isolated, the vesicles may be lysed in lysis buffer to release the cell-free nucleic acids. For example, as described Garcia, 2019, Isolation and Analysis of Plasma-Derived Exosomes in Patients With Glioma, Front Oncol, 9: 651, incorporated by reference.

The cell-free nucleic acids contained within the vesicles may comprise cell free RNA (cfRNA), which may include messenger RNA (mRNA), microRNA (miRNA), long non-coding RNA (lncRNA), and circular RNA (circRNA). The cfRNA may or may not be fragmented to a desired size. Fragmenting may be performed using sonication methods or by enzyme treatment. Preferably, the isolated cfRNA comprises a 260/280 and 260/230 absorbance ratio values of close to 2.0. Once the cfRNA are isolated, a cfRNA sample prep procedure may be performed to identify the cfRNA.

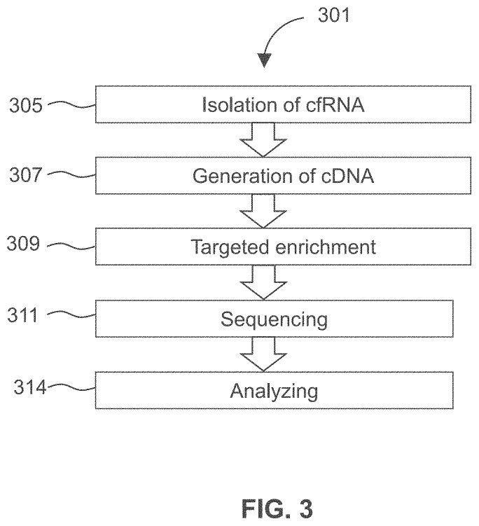

FIG. 3 diagrams a method 301 of sample prep. The method 301 includes isolating 305 cfRNA. The cfRNA is preferably isolated from extracellular vesicles collected in a blood sample. In some embodiments, RNA isolation 305 is performed with an RNA isolation kit sold, such as the RNA isolation kit sold under the trade name RNeasy by Qiagen (Valencia, Calif.), and in accordance with the manufacturer's instructions. Isolated cfRNA preferably has a 260/280 and 260/230 absorbance ratio values close to 2.0. To determine the quality of the RNA, a nucleic acid analysis system, such as the Agilent 2100 Bioanalyzer instrument, may be used. In some embodiments, the cfRNA may be chemically fragmented. Preferably, the fragments comprise 200 base pairs.

Following isolation 305, the cfRNA is converted to cDNA. The generation of cDNA 307 can be done by a variety of methods, but, preferably, the cDNA is generated using reverse transcriptase, which can use the information in a molecule of RNA to generate a molecule of cDNA. Reverse transcriptase is a RNA-dependent DNA polymerase. Like all DNA polymerases it cannot initiate synthesis de novo but depends on the presence of a primer. Since many RNAs have a poly-A tail at the 3′ end, oligo-dT is frequently used to prime DNA synthesis.

It is also possible, and frequently essential, to generate cDNAs by using either random primers or primers designed to amplify a specific RNA. Once a first strand of cDNA has been created, it is generally necessary to produce a second strand of DNA. A person of skill in the art will recognize that there are many methods for producing the second strand, but a convenient mechanism involves exposure of the DNA/RNA hybrid to a combination of RNAase-H and DNA polymerase. RNAase-H has the ability to cause single-stranded nicks in the RNA, and DNA polymerase can then use these single-stranded nicks to initiate “second strand” DNA synthesis. This two-step procedure has been optimized to maximize fidelity and length of cDNAs. In preferred embodiments, adapters are ligated onto the ends of the cDNA. The cDNA may be adenylated at the 3′ end prior to adapter ligation. Preferably, the adapters comprise sequencing platform specific primers, such as the Illumina P5/P7 (flow cell binding primers). The adapters may also comprise PCR primer biding sites for amplifying the cDNA library. In some embodiments, the adapters may further include barcode sequences. The barcode sequences may be used to give each molecule of cDNA a unique tag, e.g., a unique molecular identifier. Unique molecular identifiers or molecular barcodes are short DNA molecules which may be ligated onto DNA fragments, e.g., cDNA fragments. The random sequence composition of the unique molecular identifiers assures that every fragment-unique molecular identifier combination is unique in the library. Thus, after PCR amplification, it is possible to distinguish multiple copies of a fragment caused by PCR clones versus real biological duplications. By using unique molecular identifiers, PCR clones can be found by searching for non-unique fragment-UMI combinations, which can only be explained by PCR clones. Following adapter ligation, the cDNA may be amplified by PCR.

In preferred embodiments, biotinylated capture baits or probes are used for the targeted enrichment 309 of specific cDNA molecules of interest. The biotinylated capture probes may comprise RNA, DNA, or a hybrid of RNA and DNA nucleotides. Preferably, the capture probes comprise biotinylated RNA, which may provide better signal to noise ratios. The biotinylated RNA capture probes may be added to the cDNA library and incubated for a time, and at a temperature, sufficient for the biotinylated RNA capture probes to hybridize to their target molecules of cDNA based on Watson-Crick base pairing. For example, the mixture containing cDNA and probes may be incubated at 65 degrees Celsius for 24 hours. After hybridization, the biotinylated RNA capture probes that are hybridized with the target cDNA molecules may be captured and segregated using streptavidin or an antibody. In preferred embodiments, the target cDNA molecules are amplified by PCR.

The library may then be sequenced 311. An example of a sequencing technology that can be used is Illumina sequencing. Illumina sequencing is based on the amplification of DNA on a solid surface using fold-back PCR and anchored primers. Genomic DNA is fragmented and attached to the surface of flow cell channels. Four fluorophore-labeled, reversibly terminating nucleotides are used to perform sequential sequencing. After nucleotide incorporation, a laser is used to excite the fluorophores, an image is captured and the identity of the first base is recorded. Sequencing according to this technology is described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference. In preferred embodiments, an Illumina Mi-Seq sequencer is used. The Ilumina Mi-Seq sequencer is used to generate a plurality of sequence reads that may be uploaded to a web portal for analysis by, for example, the Agendia Data Analaysis Pipeline Tool (ADAPT).

Analyzing 314 the sequence reads may be performed using known software and following a multistep procedure known in the art. For example, first, the quality of each sequence read, i.e., FASTQ sequence, may be assessed using the software FASTQC. Next, the reads may be trimmed by, for example, Trimmomatic software. The trimmed sequence reads may then be mapped to a human genome using the HISAT2 software. HISAT2 output files in a SAM (sequence alignment/map format), which may be compressed to binary sequence alignment/map files using SAMtools version prior sequence read quantification. Afterward, mapped reads may be counted using the feature Counts software.

It may be helpful to support disease assessments made from analysis of expression levels with other data types that are indicative of disease state or progression.

One other data type that may be used in methods of the disclosure is imaging data, such as histopathology data, e.g., whole-slide imaging. Image data taken from stained tissue samples has long been used to diagnose breast cancer, including subtypes, stage, and prognoses. By combining image data with expression levels of cell free nucleic acids, a more accurate and complete picture of a patient's breast cancer can be produced.

Image data taken from stained tissues is a valuable tool for the detection and evaluation of abnormal cells such as those found in cancerous tumors. By using specific molecular markers that are characteristic of cellular events, such as, proliferation or cell death (apoptosis), a patient tissue sample can be evaluated to determine disease severity. Accordingly, methods of the invention may include obtaining an image of a stained tissue sample from the patient and analyzing the image to detect one or more features indicative of disease severity to support or confirm an assessment of disease severity or progression. The tissue sample may be obtained by biopsy. The biopsy sample may then be stained with markers that label features of disease. For example, the image may be an image of a tumor sample stained with a H&E stain, Pap stain, or any other suitable staining/labelling media. The image may be a digital scan of a stained tissue sample.

The tissue sample may comprise a tissue slice harvested from a patient. The tissue slice may contain information regarding the pathological status of the tissue. Alternatively, the tissue may comprise cells collected by, for example a biopsy, and deposited onto a slide. The cells may include any human cell type, such as, for example, lymphocytes, erythrocytes, macrophages, T-cells, skin cells, fibroblasts, epithelial cells, blood cells, etc. The tissue is imaged with, for example, a high-powered microscope to create image data.

In the methods and systems of the disclosure several features from image data may be assessed, for example, the spatial arrangements and architecture of different types of tissue elements. This can include, by way of example, global features of the epithelial and stromal regions, diversity of nuclear shape, orientation, texture, and architecture, glandular architecture, tumor infiltrating lymphocytes, lymphocyte proximity to cancer cells, the ratio of intratumoural lymphocytes to cancer cells, the tumor stroma, etc.

Methods of the disclosure may use machine learning in conjunction with expression levels to analyze breast cancer. This includes, not only providing a diagnosis or prognosis based on known expression transcript signatures, but also creating novel correlations between expression transcripts and other data. Machine learning is branch of computer science in which machine-based approaches are used to make predictions. Bera et al., 2019, Nat Rev Clin Oncol., 16(11):703-715, incorporated by reference. Machine learning-based approaches involve a system learning from data fed into it, and use this data to make and/or refine predictions. Machine learning is distinct from traditional, rule-based or statistics-based program models. Rajkomar et al., 2019, N Engl J Med, 380:1347-58, incorporated by reference. Rule-based program models require software engineers to code explicit rules, relationships, and correlations. For example, in the medical context, a physician may input a patient's symptoms and current medications into a rule-based program. In response, the program will provide a suggested treatment based upon preconfigured rules.

In contrast, and as a generalization, in machine learning a model learns from examples fed into it. Over time, the machine learning model learns from these examples and creates new models and routines based on acquired information. As a result, the machine learning model may create new correlations, relationships, routines or processes never contemplated by a human. A subset of machine learning is deep learning. Deep learning uses artificial neural networks. A deep learning network generally comprises layers of artificial neural networks. These layers may include an input layer, an output layer, and multiple hidden layers. Deep learning has been shown to learn and form relationships that exceed the capabilities of humans.

By combining the ability of machine learning, including deep learning, to develop novel routines, correlations, relationships and processes amongst vast data sets of disease biomarker features and patients' clinical data features, (e.g., expression levels and image data) the methods and systems of the disclosure can provide accurate diagnoses, prognoses, and treatment suggestions tailored to specific patients and patient groups afflicted with diseases, including breast cancer.

In some embodiments, methods of the invention exploit the correlative powers of machine learning to assess severity and progression of disease. For example, methods may include providing determined expression levels as inputs to an analysis system that is trained on training data comprising one or more sets of training expression level measurements associated with known patient outcomes. Preferably, the analysis system comprises a computer system with a machine learning algorithm. The analysis system may be a machine learning system. Any suitable machine learning system may be trained using the training data and used to analyze expression levels input into the system. The analysis system may, for example, analyze expression levels to autonomously predict disease severity or treatment outcome based on learned correlations with training expression level measurements and known outcomes.

In some embodiments, methods of the invention may further include providing an image of a stained tissue from the patient as part of the inputs to the analysis system, wherein the analysis system analyzes the image in combination with the expression levels to assess disease severity or a response to a treatment. For example, tissue images may be obtained from multiple sources and used to train a machine learning system to monitor and diagnose disease.

Methods of the invention may have applicability to deep learning networks and/or unsupervised learning networks that employ data-driven feature representation. Important clinical features of a disease may be represented at nodes within a hidden layer within such a network. Embodiments, a machine learning system is trained and then used to predict how well a given patient will respond to certain treatments. In certain aspects, the invention provides methods that include providing training data to a machine learning system. Training data includes expression levels associated with known outcomes and multiple sets of tissue images that differ in one or more aspects such as tissue type, staining technique, or image capture process. A machine learning system is then trained to recognize features associated with a disease using the training data. Methods of the invention preferably include correlating a prognosis or diagnosis of a disease from expression levels of nucleic acids derived from a patient and, in some instances, a sample tissue image (such as an image of a section from a tumor) from a patient when the machine learning system detects the features in the sample tissue image.

Methods may include generating a report that identifies indicia of disease, includes the prognosis for the cancer for the patient, include a diagnosis, or gives a prediction of a response to a treatment. A prognosis may include a probability of metastasis or recurrence. Methods of the invention may optionally include processing one or more of the images of the training data prior to providing the training data to the machine learning system, in which the processing, for example, removes noise or performs color normalization.

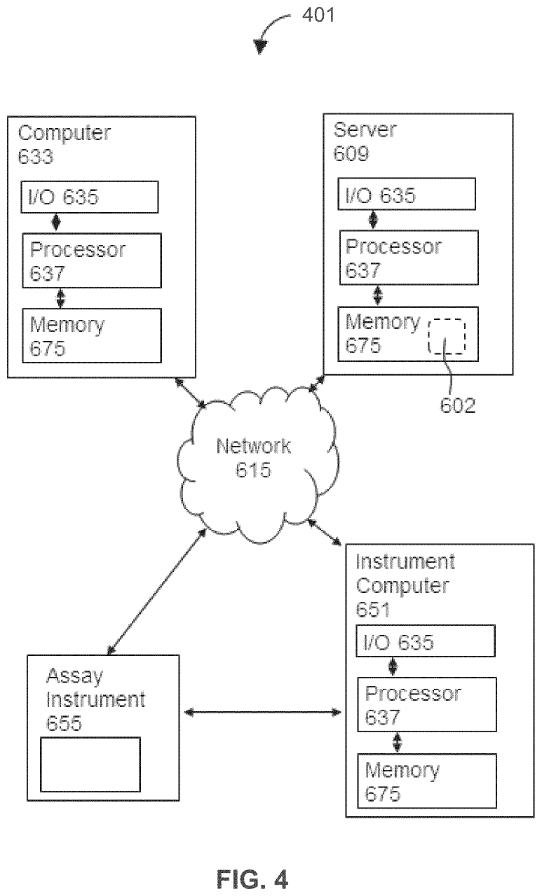

FIG. 4 shows an analysis system 401. The analysis system may include a machine learning subsystem 602 that has been trained on training data sets. In preferred embodiments, the machine learning subsystem performs the detecting 435. The system 401 includes at least one processor 637 coupled to a memory subsystem 675 including instructions executable by the processor 637 to cause the system 401 to detect 435 relevant signals; and to determine 439 a correlation to provide a predictive output.

The system 401 includes at least one computer 633. Optionally, the system 401 may further include one or more of a server computer 609 one or more assay instruments 655 (e.g., a microarray, nucleotide sequencer, an imager, etc.), which may be coupled to one or more instrument computers 651. Each computer in the system 401 includes a processor 637 coupled to a tangible, non-transitory memory 675 device and at least one input/output device 635. Thus, the system 401 includes at least one processor 637 coupled to a memory subsystem 675. The components (e.g., computer, server, instrument computers, and assay instruments) may be in communication over a network 615 that may be wired or wireless and wherein the components may be remotely located or located in close proximity to each other. Using those mechanical components, the system 201 is operable to receive or obtain training data such (e.g., images and molecular assay data) and outcome data as well as test sample data generated by one or more assay instruments or otherwise obtained. The system may use the memory to store the received data as well as the machine learning system data which may be trained and otherwise operated by the processor.

The memory subsystem 675 may contain one or any combination of memory devices. A memory device is a mechanical device that stores data or instructions in a machine-readable format. Memory may include one or more sets of instructions (e.g., software) which, when executed by one or more of the processors of the disclosed computers can accomplish some or all of the methods or functions described herein.

Using the described components, the system 401 is operable to produce a report and provide the report to a user via an input/output device. An input/output device is a mechanism or system for transferring data into or out of a computer. Exemplary input/output devices include a video display unit (e.g., a liquid crystal display (LCD) or a cathode ray tube (CRT)), a printer, an alphanumeric input device (e.g., a keyboard), a cursor control device (e.g., a mouse), a disk drive unit, a speaker, a touchscreen, an accelerometer, a microphone, a cellular radio frequency antenna, and a network interface device, which can be, for example, a network interface card (NIC), Wi-Fi card, or cellular modem. The machine learning subsystem 602 has preferably trained on training data that includes training images and known marker quantities.

Any of several suitable types of machine learning may be used for one or more steps of the disclosed methods. Suitable machine learning types may include neural networks, decision tree learning such as random forests, support vector machines (SVMs), association rule learning, inductive logic programming, regression analysis, clustering, Bayesian networks, reinforcement learning, metric learning, and genetic algorithms. One or more of the machine learning approaches (aka type or model) may be used to complete any or all of the method steps described herein.

For example, one model, such as a neural network, may be used to complete the training steps of autonomously identifying features and associating those features with certain outcomes. Once those features are learned, they may be applied to test samples by the same or different models or classifiers (e.g., a random forest, SVM, regression) for the correlating steps. In certain embodiments, features may be identified and associated with outcomes using one or more machine learning systems and the associations may then be refined using a different machine learning system. Accordingly some of the training steps may be unsupervised using unlabeled data while subsequent training steps (e.g., association refinement) may use supervised training techniques such as regression analysis using the features autonomously identified by the first machine learning system.

In decision tree learning, a model is built that predicts that value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, 2001, Random Forests, Machine Learning 45:5-32, incorporated herein by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable. Random forests can also be used to determine dissimilarity measurements between unlabeled data by constructing a random forest predictor that distinguishes the observed data from synthetic data. Id.; Shi, T., Horvath, S. (2006), Unsupervised Learning with Random Forest Predictors, Journal of Computational and Graphical Statistics, 15(1):118-138, incorporated herein by reference. Random forests can accordingly by used for unsupervised machine learning methods of the invention.

In preferred embodiments, the machine learning subsystem 602 uses a neural network. Preferably, the machine learning subsystem 602 includes a deep-learning neural network that includes an input layer, an output layer, and a plurality of hidden layers.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for assessing disease, the method comprising the steps of:
identifying at least two cell-free nucleic acids in a body fluid sample from a patient;
grouping the identified nucleic acids based on their positive predictive value for disease severity; and
using said groupings to assess disease severity.

2. The method of claim 1, wherein assessing disease comprises selecting a course of treatment for the patient, thereby to tailor a treatment to predicted disease severity.

3. The method of claim 2, wherein selecting the course of treatment comprises identifying that the patient should not receive a treatment.

4. The method of claim 1, wherein assessing disease comprises determining a response of the patient to a treatment.

5. The method of claim 4, wherein the treatment comprises a tumor resection.

6. The method of claim 2, further comprising the steps:
obtaining an image of a stained tissue sample from the patient; and
analyzing the image to detect one or more features indicative of disease severity to support or confirm the selected course of treatment.

7. The method of claim 1, wherein the cell-free nucleic acids comprise molecules of mRNA.

8. The method of claim 7, further comprising measuring quantities of the molecules of mRNA and using the measured quantities to determine expression levels for distinct species of mRNA.

9. The method of claim 8, further comprising the step of providing the determined expression levels as inputs to an analysis system that is trained on training data comprising one or more sets of training expression level measurements associated with known patient outcomes.

10. The method of claim 9, further comprising providing an image of a stained tissue from the patient as part of the inputs to the analysis system, wherein the analysis system analyzes the image in combination with the expression levels to assess disease severity or a response to a treatment.

11. The method of claim 10, wherein the analysis system comprises a computer system with a machine learning algorithm.

12. The method of claim 8, wherein the expression levels of the distinct species of mRNA are used to create one or more patient specific expression signatures for identifying aspects of disease.

13. The method of claim 12, further comprising the step of correlating one or more of the patient specific expression signatures with one or more expression signatures associated with known patient outcomes to assess likelihood of a distant metastasis event.

14. The method of claim 1, wherein the cell-free nucleic acids comprise transcripts of genes that are overexpressed in cancer patients with a 5-year survival rate greater than 75%.

15. The method of claim 1, further comprising isolating an extracellular vesicle from the blood sample and extracting molecules of RNA from the vesicle.

16. The method of claim 15, wherein the grouping step is performed exclusively on molecules of RNA that are present at a level substantially above a pre-determined threshold that is associated with background noise.

17. The method of claim 2, wherein selecting the course of treatment comprises choosing a drug.

18. The method of claim 8, wherein measuring comprises probing a panel of genes and measuring quantities of molecules of mRNA associated with the panel.

19. The method of claim 18, wherein the panel of genes includes genes involved in hormone receptor regulation.
